(19) United States
(12) Patent Application Publication
(10) Pub. No.: US 2017/0266274 A1
(43) Pub. Date: Sep. 21, 2017

(54) MUTANT PORCINE EPIZOOTIC DIARRHEA VIRUS FOR USE IN A VACCINE

(71) Applicant: Universiteit Utrecht Holding B.V., Utechi (NL)

(72) Inventors: Berend Jan Bosch, Utrecht (NL); Petrus Josephus Marie Rottier, Utrecht (NL)

(73) Assignee: Universiteit Utrecht Holding B.V., Utrecht (NL)

(21) Appl. No.: 15/502,253

(22) PCT Filed: Aug. 10, 2015
(86) PCT No.: PCT/NL2015/050571

§ 371 Date: Feb. 7, 2017

(30) Foreign Application Priority Data
Aug. 8, 2014 (EP) 14180400.5

(51) Int. Cl. A61K 39/215; C07K 14/005; C12N 7/00

(52) U.S. Cl. CPC A61K 39/215; C12N 7/00; C07K 14/005; A61K 2039/5254

(57) ABSTRACT

The invention relates to a mutant Porcine Epizootic Diarrhea Virus (PEDVdN), to methods of producing said PEDVdN, and to compositions comprising the PEDVdN. The invention further relates to methods of stimulating an immune response in a pig comprising administering the PEDVdN, to vaccines comprising the PEDVdN, and to methods for preventing or ameliorating porcine epizootic diarrhoea in a pig.

### FIELD

The present invention relates to virus-mediated animal disease, more specifically to the prevention of virus-mediated animal disease. In particular, the invention relates to the prevention of porcine epizootic diarrhea (PED). The invention relates to mutants of the causative agent, Porcine Epizootic Diarrhea Virus for vaccination of animals. The virus mutants of the invention result in an improved protection against infection with field isolates of Porcine Epizootic Diarrhea Virus.

Porcine Epidemic Diarrhea Virus (PEDV) is a lethal virus that causes a disease known as porcine epizootic diarrhea (PED) in pigs, especially piglets. PED is an acute and highly contagious enteric disease characterized by severe enteritis, vomiting and watery diarrhea. The disease is most severe in neonatal pigs as they are more susceptible to dehydration. Mortality in neonatal pigs may reach up to 100% in pigs of less than 1 week of age. PEDV was first identified in the United Kingdom in 1971, and it caused mass epidemics in Europe in the 1970s and 1980s. It has since spread to Asia, where it has been considered endemic since 1982, causing substantial economic losses to pork producers. PED was first diagnosed in the United States in May 2013, and in Canada in January 2014. The virus can spread quickly by a faecal-oral route and infect entire herds.

PEDV is a member of the order Nidovirales of the Coronaviridae. Nidoviruses employ single-stranded, polycistronic RNA genomes of positive polarity that direct the synthesis of the subunits of the replicative complex, including the RNA-dependent RNA polymerase and helicase. Replicase gene expression is under the principal control of a ribosomal frameshifting signal and a chymotrypsin-like protease, which is assisted by one or more papain-like proteases. A nested set of subgenomic RNAs is synthesized to express the 3′-proximal ORFs.

The 3′-proximal ORFs encode structural and non-structural proteins. They are expressed via a 3′-terminal nested set of subgenomic messenger RNAs. These subgenomic mRNAs encode at least four structural proteins, including three membrane anchored proteins called the spike (S), membrane (M) and envelope (E) protein, and the nucleocapsid (N) protein that encapsidates the genomic RNA. The non-structural proteins expressed from the subgenomic mRNAs encode accessory proteins which are specific for each coronavirus genus. One accessory gene, ORFS, which is located between the S and the E gene, is shared between different alphacoronaviruses and encodes a 224 amino acids (aa) long protein with three to four putative transmembrane domains (Wang et al., 2012. FEBS Lett 586: 384-391).

Vero cell-adapted strains of PEDV have been developed as candidate live attenuated vaccines. However, the resulting vaccine strains are genetically quite different from recent wild type PEDV isolates and might, therefore, not be of general use (Park et al., 2011. Arch Virol 156: 577-585; Pan et al., 2012. Virology Journal 9:195). A further disadvantage is their potential recovery of virulence and subsequent spread of virulent viruses (Chen et al., 2010. Arch Virol 155, 1471-1476). In addition, conventional inactivated vaccines are widely used, but these vaccines have some drawbacks such as high cost and sometimes poor protection efficacy (Suo et al., 2012. Virus Research 167: 259-266; Lee et al., 2012. Clin Exp Vaccine Res 1:18-34).

A vaccine that was produced using a RNA Platform vaccine technology (SirraVax®, Harrisvaccines) has recently received a conditional licensure from the United States Department of Agriculture (USDA). The RNA platform uses an Alphavirus-based expression system for expression of a spike protein of PEDV. The effectiveness of this vaccine might be limited as it expresses only the spike protein, which is known to be heterogeneous (Chen et al., 2013. Viruses 5: 2601-2613).

There is thus an urgent need for the development of novel and effective vaccines for the control of PED.

Therefore, the invention provides a mutant Porcine Epizootic Diarrhea Virus (PEDVdN), comprising a functional inactivation of a N-terminal domain of a S1 subunit of spike protein.

The spike (S) protein of alpha coronaviruses such as PEDV is a large glycoprotein of about 1255 amino acids that is anchored in the viral envelope. S proteins mediate binding of viral particles to cells, as well as cell-viral membrane fusion (Rota et al., 2003. Science 300: 1394-1399). The S protein is cleaved by host-derived proteases into two subunits: the N-terminal S1 subunit and the C-terminal membrane-anchored S2 subunit, each of which is about 150 kDa in size. The S1 subunit provides a binding domain for aminopeptidase N, a cell surface receptor for many alpha coronaviruses (Belouzard et al., 2012. Viruses 4, 1011-1033).

The length of the spike protein of alpha coronaviruses ranges from 1173 amino acid residues in human HCoV229E (SwissProt accession number P15423) to 1466 amino acid residues in feline coronavirus UU23 (GenBank accession number ADC35472.1). The sequence identity is not well conserved, showing an overall sequence identity of only 22.94% over the whole spike protein. The N-terminal region (“N-domain”) of the S1 subunit, encompassing a region from about amino acid residue 19 to amino acid residue 233 of a published sequence of S protein of a virulent strain of PEDV, as is provided in GenBank reference number JQ023161, is a less conserved region between different alpha coronaviruses, having an overall sequence identity of about 22%, compared to an overall sequence identity of about 38% for the remaining C-terminal part of the S1 subunit.

Natural variants of Transmissible GastroEnteritis Virus (TGEV) and Feline Infectious Peritonitis Virus (FIPV) type I, which are both alpha coronaviruses, have been identified in which the he N-domain of the S1 subunit was found to be absent.

Porcine Respiratory Corona Virus (PRCV) is a natural variant of TGEV and has a deletion of the N-domain of the S1 subunit. PRCV does not infect intestinal cells and, therefore, does not cause diarrhoea. PRCV multiplies mainly in the lungs, suggesting that the N-domain of the S1 subunit plays a role in interaction with a host cell receptor. It is thought that PRCV has naturally vaccinated the swine herds as PRCV infections may result in the induction of antibodies that are cross-reactive with TGEV. Both TGEV and PRCV are able to bind aminopeptidase N, and the aminopeptidase N-binding domain was found to be present in the C-terminal part of the S1 subunit at amino acid positions 481 to 650 of TGEV S protein (Reguera et al., 2012. PLoS Pathogens 8, e1002859).

In addition, a type I FIPV with a large deletion of 735 bp within the N-terminal region of the spike protein has been observed as a spontaneous mutant of FIPV. Notably, in this case the mutant virus retains its virulence and was found to induce feline infectious peritonitis in infected cats (Terada et al., 2012. J Gen Virol 93: 1930-4). Type I feline coronavirus is known not to recognize aminopeptidase N as a functional receptor on feline cell lines (Dye et al., 2007. J Gen Virol 88: 1753-60). This not only suggests that the receptor-binding domain is present in the C-terminal part of the S1 subunit, but also that deletion of the N-terminal region of the spike protein does not impact infectivity and virulence of this coronavirus.

It has been found that the aminopeptidase N-binding domain of PEDV is present in the N-terminal part of the S1 subunit, including amino acid residues 25-88 (Lee et al., 2011. Korean J Microbiol Biotechnol 39: 140-145). It was therefore surprising that a mutant PEDV, comprising a functional inactivation of the N-terminal domain of a S1 subunit of spike protein including the putative aminopeptidase N-binding domain of PEDV, was able to infect cells and spread in a target organism. In addition, said mutant PEDV was found to be attenuated, thereby impacting virulence of this coronavirus.

That the N-terminal region of the S1 spike subunit of coronaviruses is less conserved might reflect the fact that different coronaviruses may interact with different parts of the aminopeptidase N (APN) protein. For example, it has been reported that HCV 229E interacts with the amino-terminal part of APN proteins, while TGEV and canine coronavirus interact with the carboxy-terminal part of APN proteins (Kolb et al., 1997. J Gen Virol 78: 2795-2802). In addition, the receptor-binding domain of the prototype beta-coronavirus murine hepatitis virus was found to reside in the N-terminal part of the S1 subunit (Mou et al., 2013. J Virol 87: 9379-9383). Therefore, the N-terminal part of the S1 subunit of PEDV might interact with a domain on APN protein that differs from, or only partly overlaps with, the interaction domains of HCV 229E, TGEV and/or canine coronavirus.

The terms “functional inactivation” and “functionally inactivated”, as used herein, refer to a gene or part of a gene that is not expressed because it is not transcribed and/or not translated, for example by alteration or deletion of one or more nucleotides within the coding region of the gene. The term “functionally inactivated” preferably refers to a gene of which at least a part or all of the coding sequences has been deleted.

The term “N-terminal domain of a S1 subunit of spike protein”, as used herein, refers to a region corresponding to a region from about amino acid residue 19 to about amino acid residue 233 of SEQ ID NO:1. The genomic region that encodes the N-terminal domain of a S1 subunit of spike protein is conserved between different published PEDV genomic sequences, having an overall identity of about 22%. The term “corresponding to”, as used herein, indicates that an amino acid region from other PEDV need not be identical in sequence and/or numbering to the indicated region of SEQ ID NO:1. However, the skilled person will be able to determine a corresponding region in other PEDV, based on, for example, the alignment of sequences from multiple PEDV genomic sequences. The person skilled in the art will therefore understand that the amino acid residue identity and/or numbering as applied herein is not limiting for the invention, but is only applied for clarity purposes.

A PEDVdN according to the invention preferably functionally expresses a C-terminal domain of the S1 subunit of spike protein. Said C-terminal domain starts at the amino acid sequence X-X-N-V-F at position 234 of SEQ ID NO:1, or at a corresponding position in other PEDV sequences. The conserved residues N-V-F will assist a person skilled in the art to determine a corresponding position in a S1 subunit of spike protein of a different strain of PEDV.

Said PEDVdN preferably comprises genomic sequences encoding the 10 most N-terminal amino acids, preferably the 14 most N-terminal amino acids, preferably the 18 most N-terminal amino acids, which constitute a putative signal peptide.

Said PEDVdN preferably comprises a deletion of a genomic region that encodes at least three amino acids of or within the N-terminal domain of the S1 subunit of the spike protein. Said at least three amino acids are preferably three consecutive amino acid residues that constitute an antigenic region against which antibodies are generated after infection of a porcine with PEDV. Said at least three amino acids preferably encompasses amino acid residues 194-196 (amino acid sequence NKR) of SEQ ID NO:1, or corresponding amino acid residues in other PEDV sequences.

The invention therefore provides a mutant Porcine Epizootic Diarrhea Virus (PEDVdN), comprising a deletion of three or more amino acid residues within a region corresponding to a region from about amino acid residue 19 to about amino acid residue 233 of SEQ ID NO:1.

The term “deletion”, as is used herein, refers to the removal of a genomic region, preferably a genomic region that encodes at least three amino acids within the N-terminal domain of the S1 subunit of the spike protein, preferably within to a region corresponding to a region from about amino acid residue 19 to about amino acid residue 233 of SEQ ID NO:1. Said deletion results in the reduction of the number of amino acid residues in the region corresponding to a region from about amino acid residue 19 to about amino acid residue 233 of SEQ ID NO:1.

A preferred PEDVdN comprises a deletion of a genomic region that encodes amino acid (aa) 19-aa 233 of a N-terminal domain of a S1 subunit of spike protein. Said deletion is in frame, meaning that the genomic region encodes a protein in which the 18 most N-terminal amino acids of the spike S1 subunit are fused to the C-terminal part of the spike S1 subunit. Said fusion may be direct, meaning that the 18 most N-terminal amino acids of the spike S1 subunit are immediately N-terminal to the C-terminal part of the spike S1 subunit without any intervening amino acids. Alternatively, said fusion is indirect, meaning that one or more amino acid residues are positioned between the 18 most N-terminal amino acids of the spike S1 subunit and the C-terminal part of the spike S1 subunit. The coding sequence for the one or more amino acid residues that are positioned between the 18 most N-terminal amino acids and the C-terminal part of the spike S1 subunit may be any coding sequence for at least one amino acid residue.

It has been reported that vaccine strains may differ from recently isolated wild type PEDV strains (Park et al., 2011. Arch Virol 156: 577-585; Pan et al., 2012. Virology Journal 9:195). They may further differ in their mode of delivery, in their efficacy, and in their genomic sequence. Therefore, to provide efficient protection against subsequent infection with a virulent PEDV, the viral genomic sequences of PEDVdN, particularly those encoding the most immunodominant protein, i.e. the spike protein, are preferably of a contemporary virulent PEDV. Especially sequences encoding the C-terminal part of the S1 subunit of spike protein are of a contemporary virulent contemporary PEDV, as this part stimulates the induction of neutralizing antibodies most strongly and hence is an important target in developing effective vaccines.

A PEDVdN according to the invention is preferably attenuated by providing PEDVdN with a deletion in, preferably of, one or more genes not being genes specifying the polymerase functions (ORF 1a/1b) or the structural proteins N, M, E, and S. Said gene preferably is ORF3, preferably said attenuated virus comprises a deletion of ORF3. It has been indicated, though not demonstrated, that mutations in the ORF3 gene resulting from cell culture adaptation may contribute to the attenuation of these viruses (Song et al., 2007. Res Vet Sci 82: 134-140). In addition, siRNA-mediated knockdown of ORF3 gene in PEDV infected cells reduced the number of particles released from the cells (Wang et al., 2012. FEBS Lett 586: 384-391). Deletion of the complete 224 amino acids long ORF3 open reading frame (Li et al., 2013. PlosOne 8: e6997) suggested that this deletion may hamper propagation and/or shedding of the virus in vivo, thereby attenuating the virus.

A PEDVdN according to the invention is preferably attenuated by generation of mutant viruses according to the invention with rearranged gene order, as described in WO2002/092827 for FIPV. For example, the position of the M and/or E gene on the viral genome can be altered, for example by positioning the open reading frames of the M and/or E gene in front of the ORF3 gene, preferably in front of the S gene. For this, restriction sites are preferably engineered upstream of the ORF3 gene, in which the coding sequences of the M and/or E gene are inserted. The principle of this method is detailed in WO2002/092827, which is incorporated herein by reference. Suitable methods for introducing restriction sites, and for amplification of M and/or E gene are known, for example as described in Green and Sambrook, “Molecular Cloning: A Laboratory Manual”, CSHL Press, 2012. It is preferred that the resulting constructs are confirmed by restriction and/or sequence analysis, prior to the generation of recombinant viruses by RNA-RNA recombination between transcription vector run-off transcripts and the PEDV genome. A PEDVdN according to the invention is preferably attenuated by a deletion in, preferably of, one or more genes not being genes specifying the polymerase functions (ORF 1a/1b) or the structural proteins N, M, E, and S, preferably deletion of the ORF3 gene, in combination with a rearranged gene order, as described in WO2002/092827 for FIPV.

PEDVdN according to the invention preferably comprises a marker gene. Said marker gene includes, but is not limited to, a Fluorescent Protein, for example Green Fluorescent Protein, beta-glucuronidase, Renilla luciferase, and beta-galactosidase. Said marker gene will allow to identify cells and/or animals that are infected with PEDVdN.

A PEDVdN comprising a functional inactivation of a N-terminal domain of a S1 subunit of spike protein according to the invention, preferably is a live, infectious virus. In general, immunization by a live, attenuated vaccine typically is more effective than by a killed or inactivated vaccine. A live, infectious virus, for example, provides a strong, long-lasting immune response that is achieved with fewer doses, does not require adjuvants, may quickly stimulate non-specific, antiviral protection via interferon production, and uses the same mode of delivery as the disease-causing form of the virus. However, an inactivated PEDVdN may be more stable on storage and is unlikely to cause disease due to residual disease-causing characteristics.

The invention further provides a cell comprising the PEDVdN according to the invention. Said cell preferably is used for propagation of the virus. A preferred cell is an eukaryotic cell, preferably a cell that can easily be infected using standard methods known to the skilled person. Said cell is preferably a mammalian cell. Suitable cells comprise, for example, Baby Hamster Kidney cells such as BHK-21, Human Embryonic Kidney cells such as HEK293, VERO cells (ATCC® CCL-81™), MDCK cells, CHO cells, Huh-7, Huh7.5 (Sumpter 2005. J Virol 79:2689-2699), HeLa, and L cells (ATCC® CRL-2648™). Preferred cells are VERO cells and/or L-cells.

Propagation of PEDV in cell culture may require addition of trypsin, which is believed to prime or activate the S protein for membrane fusion during virus cell entry and syncytia formation (Hofmann and Wyler, 1988. J Clin Microbiol 26: 2235-2239). However, cell culture adapted strains have been reported which are able to replicate in the absence of trypsin (Kweon et al., 1999. Vaccine 17: 2546-2553).

The invention also provides a method of producing PEDVdN according to the invention, comprising providing a cell with a RNA molecule encoding the PEDVdN. For this, a RNA recombination system preferably is employed in which a recombinant hybrid PEDV virus carrying a genomic sequence encoding a spike protein from a coronavirus that infects non-porcine animals, for example from a mouse hepatitis virus (MHV), is constructed as described (Li et al., 2013. PlosOne 8, e69996). In a first stage, recombinant progeny hybrid PEDV viruses are selected on non-porcine, for example mouse, cells, on the basis of the acquired ability to form plaques in murine cell monolayers. Recombinant progeny PEDV viruses comprising a functional inactivation of a N-terminal domain of a S1 subunit of spike protein are subsequently selected in a second stage on the basis of their ability to infect VERO cells and the concomitantly lost ability to infect murine cells.

The invention further provides a composition comprising a PEDVdN according to the invention and a pharmacologically and/or veterinary acceptable carrier. Said pharmacologically and/or veterinary acceptable carrier may comprise vitamins; sugars such as sucrose, lactose, D-mannose, D-fructose, and/or dextrose; amino acids such as, for example, glycerin and asparagine; inorganic salts such as, for example, sodium bicarbonate, aluminum hydroxide, benzethonium chloride, ammonium sulfate, magnesium sulfate, potassium phosphate, sodium phosphate, aluminum phosphate and aluminum potassium sulfate; micro crystalline cellulose, magnesium stearate, cellulose acetate phthalate, human serum albumin, fetal bovine serum, citric acid, iron ammonium citrate, peptone, bovine extract and/or gelatin.

The composition according to the invention preferably is an immunogenic composition, more preferably a composition that provides protection against a subsequent infection with a wild type PEDV. Said protection against wildtype virus is characterized as a reduction of clinical disease, and/or a reduction of replication of wild type virus in the host.

Inactivated PEDVdN may be generated by killing of the virus, typically using heat or chemicals such as formaldehyde or formalin. Inactivated viruses are generally administered together with an adjuvant and/or a carrier or other excipient known in the art. An adjuvant, as defined herein, is a substance that is added to an immunological composition in order to enhance the immune response of an individual to a particular antigen at either the cellular of humoral level. Preferred adjuvants include, but are not limited to, Stimune (Specol)®, TiterMax®, Montanides, MF-59, AS03, QS21, Adjuvant 65, saponin, MDP, Syntex Adjuvant Formulation (SAF)®, monophosphoryl lipid A, Gerbu® Adjuvant, immune-stimulating complexes (ISCOMs) and bacterium-like particles (BLPs; van Braeckel-Budimir et al., Frontiers in Immunology 4, Article 282, 2013). An immunological composition comprising an inactived PEDVdN preferably further comprises cytokines such as Interferon-gamma, immunostimulatory nucleic acid sequences such as CpG oligonucleotides, liposomes, virus-like particles, surfactants such as hexadecylamine, polyanions such as pyran and dextran sulfate.

A preferred adjuvant is ISCOM, as described in the international patent application WO2002026255A1. ISCOM technology has several advantages over other adjuvants. ISCOMs stimulate both humoral and cell-mediated immune responses. ISCOMs is a highly efficient adjuvant, enabling further reduction of the amounts of an inactivated or part thereof according to the invention. A preferred ISCOM is ISCOM Matrix-M.

Another preferred adjuvant is BLP. BLPs are self-adjuvanting vaccine delivery vehicles, derived from inactivated Lactococcus lactis bacteria. L. lactis is a safe bacterium commonly used in the food industry, such as for the production of cheese and probiotic drinks. BLPs are produced by a simple hot acid treatment, resulting in a robust cell shaped matrix that predominantly consists of a peptidoglycan surface. Said peptidoglycan preferably comprises the C-terminal peptidoglycan binding domain LysM of Lactococcus lactis cell wall hydrolase AcmA as described in WO 2010/033031. This surface induces a long-lasting immunity needed for protection against disease causing pathogens. The non-living nature of BLP particles allows for accurate dosing without a risk of dissemination.

The BLPs also provide a safe and versatile backbone that can be efficiently loaded with particular antigens of choice, for example a mutant PEDVdN according to the invention. Complete loading of BLPs with antigens is achieved by using a non-covalent coupling technology as is described in WO2010/033031. This technology allows for simple mixing of the antigen fusion with the BLPs, thereby resulting in robust and immediate binding of the antigen to the surface of the particles. The resulting antigen-covered BLPs is preferably delivered to a mammal such as a pig via the mucosal layers of the nose (spray) or mouth (capsule), without a need for an injection.

The invention further provides a method of stimulating an immune response in a pig comprising administering a composition comprising a PEDVdN according to the invention to the pig in an amount effective to induce an immune response. The pharmaceutical composition comprising said PEDVdN, inactivated or not, may be administered orally, parenterally, by inhalation, or through other routes. Oral or, preferably, oronasal administration can be performed in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch.

Preferably the PEDVdN, inactivated or not, is administered parenterally, preferably by injection. The virus, inactivated or not, according to the invention is preferably formulated with conventional, non-toxic pharmaceutically-acceptable carriers, adjuvants or vehicles. The term parenteral, as used herein, includes subcutaneous, intracutaneous, intravenous, intramuscular, intra-articular, intrasynovial, intrasternal, intrathecal, intralesional and intracranial injection or infusion techniques. A most preferred administration route is subcutaneous injection. Inactivated PEDVdN comprising BLP as adjuvant is preferably oronasally administered via the mucosal layers of the nose, for example by using a spray, and/or to the mouth, for example by a capsule or by using a spray.

The PEDVdN, inactivated or not, is preferably in the form of an injectable preparation, for example, an injectable aqueous or oleaginous suspension. This suspension may be formulated according to techniques known in the art using suitable dispersing or wetting agents (such as, for example, Tween 80) and suspending agents. The injectable preparation may also be an injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or a similar alcohol as described in the Pharmacopoea Helvetica.

The amount of vaccine virus of the invention that is administered to an animal is, in general, in the range of 1,000 to 1,000,000,000 infectious virus particles per ml. The amount of infectious particles can be determined using standard techniques known to the skilled person such as, for example, a dose response curve.

The invention further provides a vaccine, preferably a Differentiating Infected animals from Vaccinated Animals (DIVA) vaccine, comprising an effective immunizing amount of the composition comprising a PEDVdN according to the invention. Said vaccine provides protection against a subsequent infection with a wild type, virulent PEDV. Protection is defined as reduction of onward spread of wild type virus by any transmission route, including horizontal and vertical spread. The time to onset of protection and long lasting protection are part of the efficacy of a vaccine. Further, broad protection against different virus species or serotypes is also part of efficacy of a vaccine according to the invention. The term DIVA is used for vaccines in conjunction with a differentiating diagnostic test. This system enables the mass vaccination of a susceptible animal population without compromising the serological identification of convalescent animals.

It was found that animals that were infected by PEDV raise a humoral response directed against the N-terminal domain of a S1 subunit of spike protein. A PEDVdN according to the invention, therefore, allows differentiation between vaccinated and infected animals based on the humoral response against the N-terminal domain of a S1 subunit of spike protein.

A relevant DIVA-associated assay comprises a peptide that comprises the N-terminal domain of a S1 subunit of spike protein, or a relevant part thereof. Said assay preferably is an antibody enzyme-linked immunosorbent assay (ELISA), allowing diagnosis of PEDV in pigs. Preferred ELISAs are sandwich type ELISAs. A preferred ELISA is a competition ELISA based on the N-terminal domain of a S1 subunit of spike protein. Said DIVA-associated assay preferably further comprises one or more antibodies such as monoclonal antibodies, or antibody-like proteins, that recognize the N-terminal domain of a S1 subunit of spike protein.

Further contemplated herein is a method of differentiating animals, preferably pigs, infected with PEDV from non-infected animals or from animals vaccinated with a PEDVdN-based vaccine according to the invention, comprising analyzing serum of the animal in a relevant DIVA-associated assay as herein described.

As an alternative, or in addition, another DIVA-associated method is based on determining the presence or absence of the N-domain of the spike S1 subunit, or of a nucleotide sequence encoding said N-domain of the spike S1 subunit, in a sample of an infected animal, for example in a blood sample or in a fecal sample.

Methods for determining presence or absence of the N-domain of the spike S1 subunit are known, including antibody-mediated detection methods such as Enzyme-Linked Immuno Sorbent Assay (ELISA).

Methods for determining presence or absence of a nucleotide sequence encoding said N-domain of the spike S1 subunit, or a part thereof, include amplification of a nucleic acid molecule, for example by Polymerase Chain Reaction (PCR), rolling circle amplification, nucleic acid sequence-based amplification, transcription mediated amplification, and/or linear RNA amplification, using a set of primers that flank said nucleotide sequence encoding said N-domain of the spike S1 subunit, or a part thereof. The amplified product may be directly stained or labelled with radioactive labels, antibodies, luminescent dyes, fluorescent dyes, or enzyme reagents. Direct DNA stains include for example intercalating dyes such as SYBR® Green (Life Technologies Corporation), acridine orange, ethidium bromide, ethidium monoazide or Hoechst dyes. The set of primers preferably includes a forward primer and a reverse primer, as is known to a skilled person, which are single-stranded oligonucleotides or oligonucleotide mimics of 15-50 bases, preferably 16-30 bases, that are complementary to nucleic acid sequences flanking the region to be amplified. The sequence of the forward primer and reverse primer determine the specificity of the amplification reaction. Preferred primers are preferably about 100% identical to a region on a target nucleic acid template such that only the region between two primers in a target nucleic acid template is amplified. The distance between the primer binding sites on the target nucleic acid template will determine the size of the amplified product.

The invention further provides a method for preventing or ameliorating porcine epizootic diarrhea in a pig, comprising administering a PEDVdN according to the invention, preferably formulated as an immunogenic composition or a vaccine, to the pig.

The term pig, as used herein, refers to an animal in the Suidae family of even-toed ungulates. The term pig includes a domestic pig and its ancestor, the common Eurasian wild boar (Sus scrofa), Palawan bearded pig, Bornean bearded pig, Heude's pig or Vietnamese warty pig, Visayan warty pig, Celebes warty pig, Flores warty pig, Mindoro warty pig, Philippine warty pig, Java warty pig, babirusa and warthog.

The ability to raise antibodies to an infection virus is weak in piglets, especially in piglets of 0-7 days of age. Active adaptive immunity must develop rapidly and appropriately in a neonate because immune protection acquired by the fetus from the mother via placental transfer, colostrum, and breast milk does not confer protection against antigens to which the mother has not been exposed. An effective immune responses in general in piglets develops in piglets between 1 and 2 weeks of age. Piglets of about 4 weeks of age become fully responsive to invading antigens, including viruses such as PEDV.

Therefore, a PEDVdN according to the invention may be administered to a pig, such as a piglet. Said PEDVdN preferably is administered to a pregnant sow. Passive transfer of especially immunoglobulin A from an immunized sow via placental transfer, colostrum, and breast milk will effectively provide protection against invading antigens, including viruses such as PEDV, in newborn piglets.

### FIGURE LEGENDS

FIG. 1. Spike protein sequences of the PEDV-Sca_flag and PEDV-ScaΔN_flag viruses.

1A: Sca_flag protein sequence. The 18 most N-terminal amino acids are in bold, while the N-terminal amino acids A-A-N-V-F of the C-terminal part of the S1 subunit are underlined, the Flag-tag containing sequence is shown in italics.

1B: ScaΔN flag protein sequence. The 18 most N-terminal amino acids are in bold, while the N-terminal amino acids A-A-N-V-F of the C-terminal part of the S1 subunit are underlined, the Flag-tag containing sequence is shown in italics.

FIG. 2. Spike nucleotide sequences of the PEDV-Sca_flag and PEDV-ScaΔN_flag viruses.

2A: Sca_flag_nucleotide_sequence. The sequence encoding the 18 most N-terminal amino acids is in bold, while that of the N-terminal amino acids A-A-N-V-F of the C-terminal part of the S1 subunit is underlined, the Flag-tag containing sequence is shown in italics.

2B: ScaΔN_flag_nucleotide_sequence. The sequence encoding the 18 most N-terminal amino acids are in bold, while that of the N-terminal amino acids A-A-N-V-F of the C-terminal part of the S1 subunit are underlined, the Flag-tag containing sequence is shown in italics.

FIG. 3. Nucleotide sequence of part of the PEDV-ScaΔN_flag virus genome covering the Spike N-domain deletion.

PEDV-ScaΔN_flag_nucleotide_sequence. The sequence encoding the 18 most N-terminal amino acids of the Spike protein are in bold, while that of the N-terminal amino acids A-A-N-V-F of the C-terminal part of the S1 subunit are underlined.

FIG. 4. Outline of spike protein and of ΔN spike protein. Potential glycosylation sites are indicated (Ψ).

FIG. 5. Recombinant PEDV carrying S lacking N-domain is viable. Fluorescence images of cells infected with the GFP-expressing recombinant PEDV-Sca_flag and PEDV-ScaΔN_flag virus.

FIG. 6. Recombinant PEDV carrying S lacking N-domain is viable. Comparative growth curve of PEDV-Sca_flag and PEDV-ScaΔN_flag viruses. Vero cells were inoculated with the indicated viruses. The tissue culture infective dose that produces a pathological change in 50% of cell cultures inoculated (TCID50) was determined in the culture medium at the indicated times.

FIG. 7: Western blot analysis of the S proteins on the PEDV-Sca_flag and PEDV-ScaΔN_flag recombinant viruses. Semi-purified virions were subjected to Western blotting and S proteins were detected by ECL chemiluminescence imaging using an antibody directed against the C-terminally appended flag-tag. Position and size (in kDa) of the protein molecular weight markers are indicated on the left.

### EXAMPLES

### Example 1

### Construction of Recombinant Viruses

The transfer vector for the PEDV-ScaΔN_flag virus was a derivative of the previously described PEDV-Sca_flag transfer vector, the latter containing i) a BamHI restriction site between ORF1b and the S gene, ii) a gene encoding the green fluorescent protein as a replacement of the ORFS gene and iii) a FLAG peptide (DYKDDDDK)-encoding gene fragment as a C-terminal extension of the S gene (Li et al., 2013. PlosOne 8: e6997).

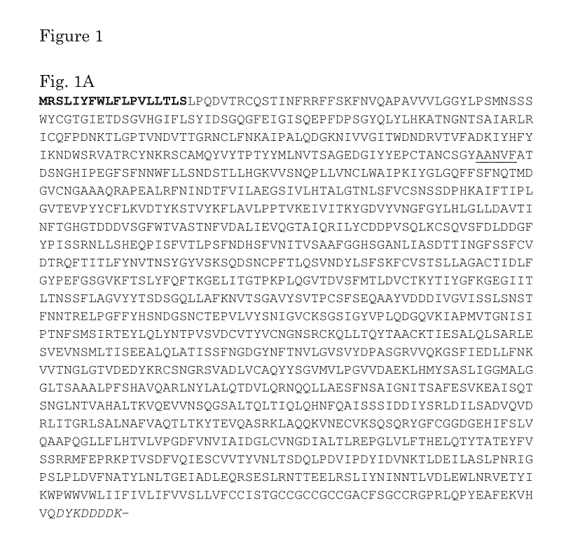

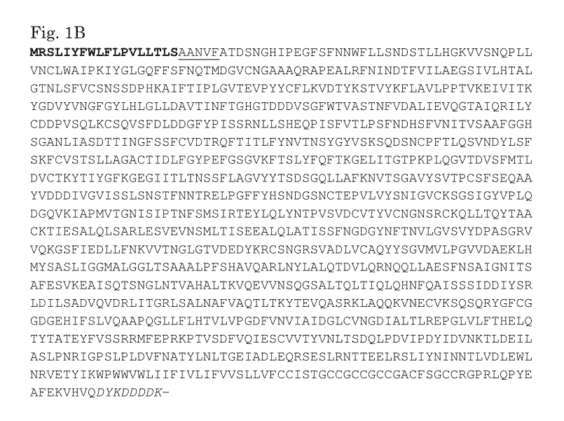

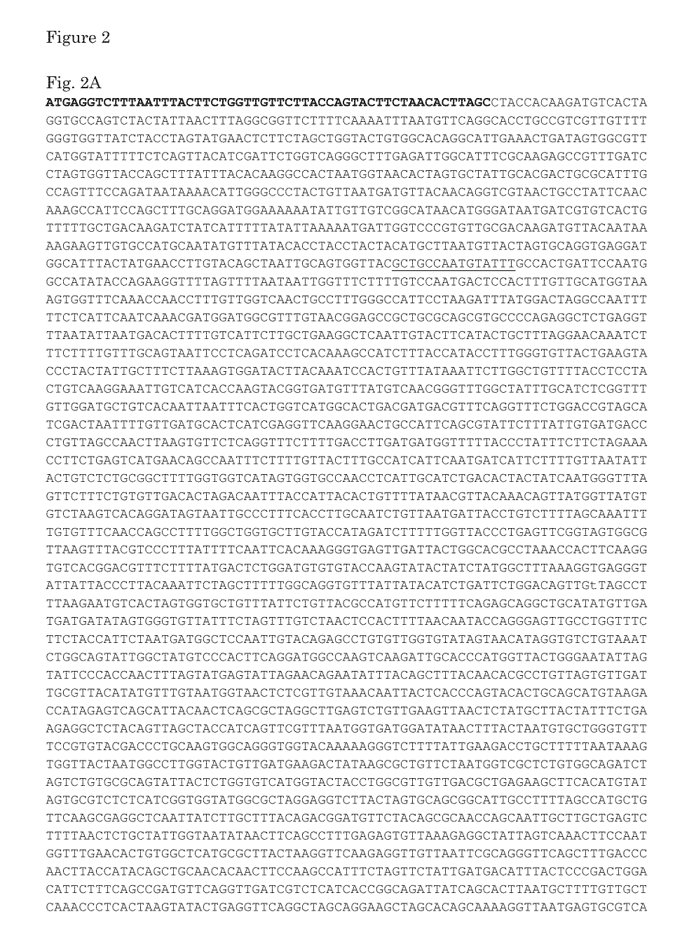

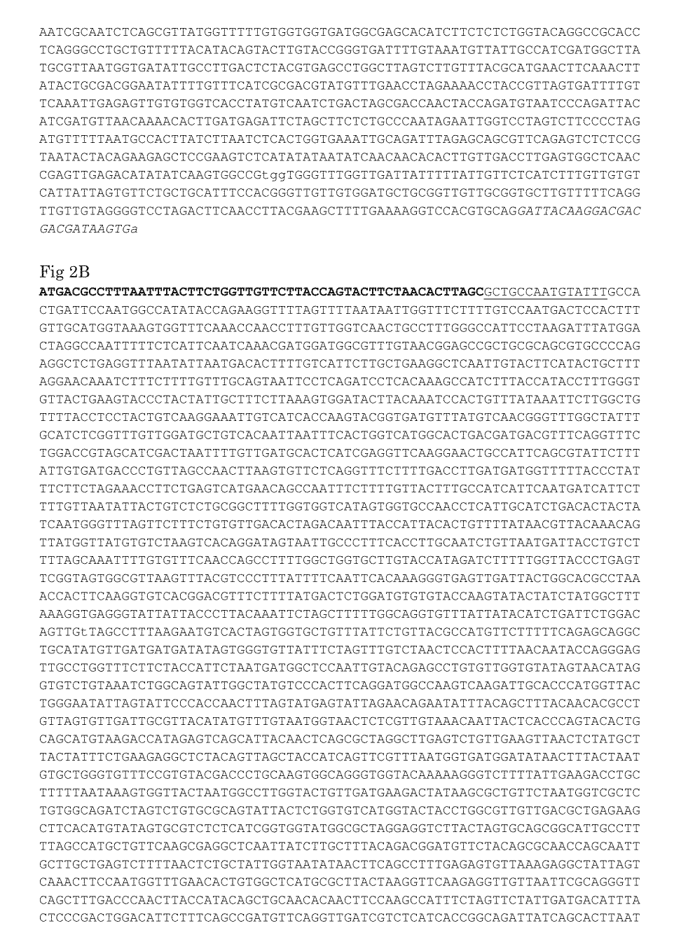

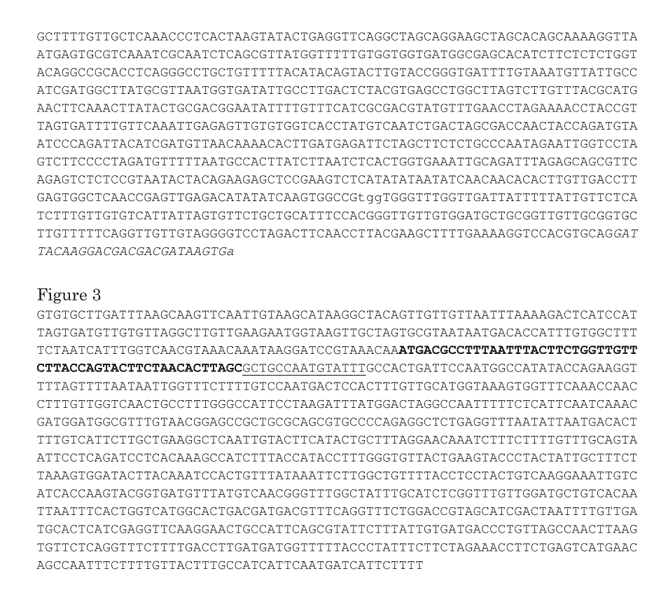

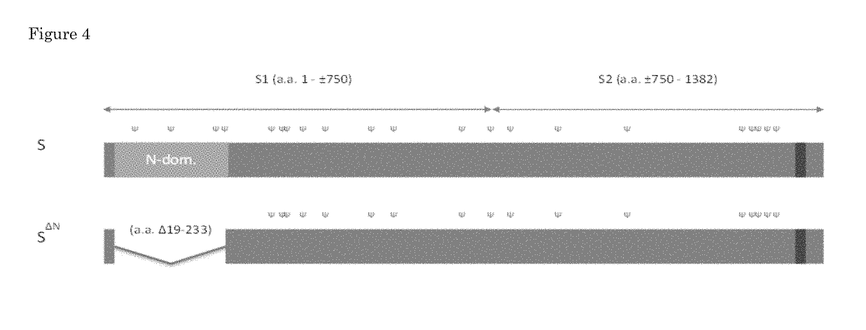

The PEDV-ScaΔN_flag transfer vector with the S gene lacking the sequence encoding the S-N-domain (residues 19-233, FIGS. 1, 2, 3 and 4) was constructed by in-fusion cloning using primer pair 8206 and 8207 (5′-GCTGCCAATGTATTTGCC-3′ and 5′-GCTAAGTGTTAGAAGTACTG-3′, resp.), using the PEDV-Sca_flag transfer vector as a template.

The PEDV-ScaΔN_flag virus was generated as described by Li et al. (Li et al., 2013. PlosOne 8: e6997) and Wicht et al (Wicht et al. JVI 2014). Viral RNA was extracted from the PEDV-ScaΔN_flag virus at two to three passages after plaque selection, and the genotype was confirmed by sequencing using primer 5109 (5′- GACGGCAACACCATGCATGCC-3′).

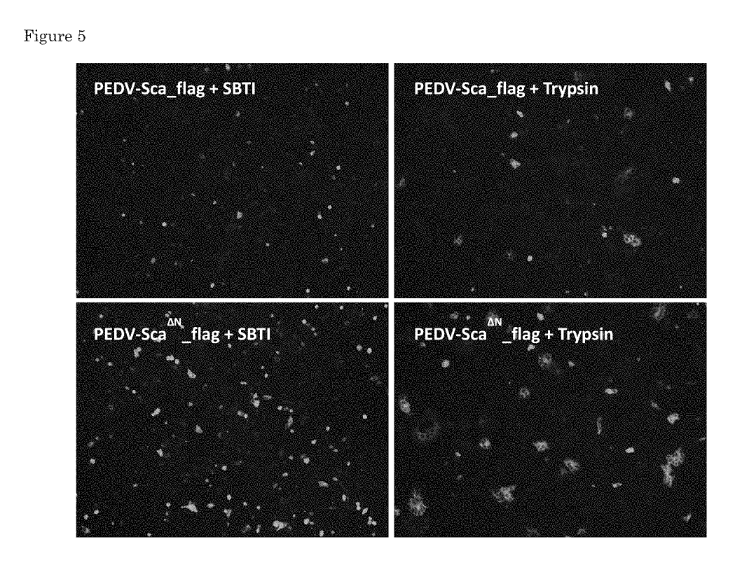

Vero cells were transiently transfected with expression plasmids encoding the PEDV-Sca_flag and PEDV-ScaΔN_flag protein for 48 h. Cells were treated with trypsin or soybean trypsin inhibitor (SBTI) for 1 h and subsequently examined by immunostaining against S protein. As is shown in FIG. 5, PEDV-ScaΔN_flag protein has the capacity to mediate cell-cell fusion upon exposure to trypsin.

### Example 2

Vero cells were inoculated for 1 hour at an MOI of 0.1 with recombinant strain DR13-based PEDV in which the ORFS gene had been replaced by a GFP gene and which carried a Flag-tagged S protein (rPEDV-SDR13_Flag-dORF3/GFP) (see Li et al., 2013. PlosOne 8: e6997). In the virus rPEDV-SDR13-dN_Flag-dORF3/GFP, the N-domain of the S protein had been deleted.

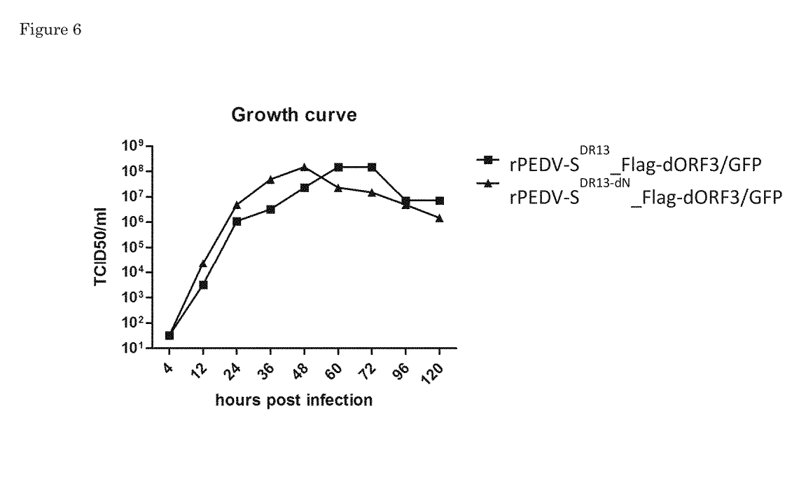

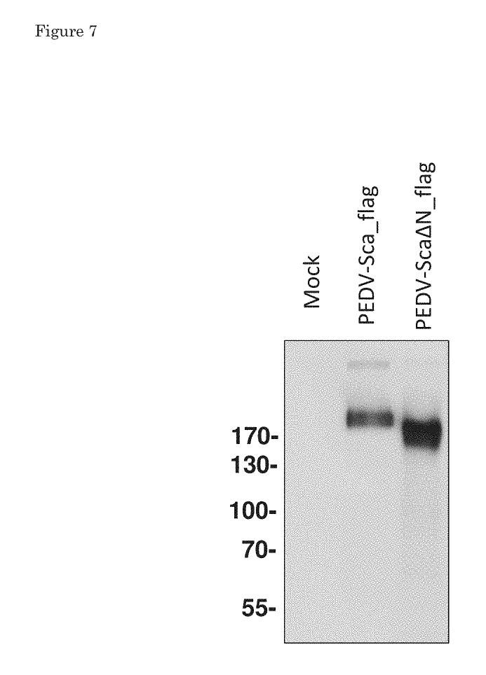

Small aliquots were taken from the culture medium every 12 hours and the infectivity released from the cells into the medium was determined by titration (TCID50) of the virus in these samples on Vero cells. As is indicated in FIG. 6, the growth curve of rPEDV-SDR13-dN_Flag-dORF3/GFP is comparable to the growth curve of rPEDV-SDR13_Flag-dORF3/GFP and both viruses grow to high titers.

### Example 3

### Material and Methods

Vero cells were inoculated for 1 hour at an MOI of 1 with PEDV-Sca_flag and PEDV-ScaΔN_flag. At 24 h post infection, virus containing cell culture supernatants were collected and virus particles in the supernatant were concentrated by sedimentation through a 20% cushion of sucrose in HCN buffer (50 mM HEPES, 100 mM NaCl, 10 mM CaCl2) at 100,000×g for 3 h at 4° C. A similar purification procedure was done with culture medium from mock infected Vero cells (Mock). Virus particles were handled on ice and resuspended in HCN buffer. Samples were subjected to sodium dodecyl sulfate-polyacrylamide gel electrophoresis (SDS-PAGE) in a discontinuous gel with 8% acryl amide in the separating gel. Next, samples were transferred to a polyvinylidene fluoride membrane (BioRad, 162-0176) and blocked with bovine serum. The flag-tagged PEDV S proteins were detected using a mouse monoclonal anti-FLAG conjugated to horseradish peroxidase (Sigma, A8592) with a swine anti-rabbit immunoglobulin G conjugated horseradish peroxidase (Dako, P0217) as a secondary antibody. Protein bands were visualized by Chemiluminescent autoradiography using the Amersham ECL Western Blotting Analysis System (GE healthcare, RPN2109) in combination with X-Omat LS films (Kodak, Sigma F1149).

### Results

The S proteins of PEDV-Sca_flag and PEDV-ScaΔN_flag recombinant viruses were analysed biochemically by Western Blotting using an antibody that recognizes the C-terminal flag-tag. The results indicate a faster gel mobility of the S protein of the PEDV-ScaΔN_flag virus compared to that of the PEDV-Sca_flag virus, which is consistent with the deletion of most of the N-domain of the spike protein.

1. A mutant Porcine Epizootic Diarrhea Virus (PEDVdN), comprising a deletion of three or more amino acids residues within a region corresponding to a region from about amino acid residue 19 to about amino acid residue 233 of SEQ ID NO:1.

2. The PEDVdN of claim 1, which functionally expresses a C-terminal domain of the S1 subunit of spike protein.

3. The PEDVdN of claim 1, wherein the deletion encompasses amino acid residues 194-196, having the amino acid sequence NKR, of SEQ ID NO:1, or corresponding amino acid residues in other PEDV sequences.

4. The PEDVdN of claim 1, wherein all amino acid residues are deleted within the region corresponding to the region from about amino acid residue 19 to about amino acid residue 233 of SEQ ID NO:1.

5. The PEDVdN of claim 1, wherein the viral genomic sequences are of a virulent PEDV.

6. The PEDVdN of claim 1, wherein the virus is attenuated by a deletion in ORF3, preferably a deletion of ORF3, and/or by a rearranged gene order.

7. The PEDVdN of claim 1, further comprising a marker gene.

8. The PEDVdN of claim 1, which is a live, infectious virus.

9. A cell comprising the PEDVdN of claim 1.

10. A method of producing PEDVdN of claim 1, comprising providing a cell with a RNA molecule encoding the PEDVdN.

11. A composition comprising the PEDVdN of claim 1 and a pharmacologically and/or veterinary acceptable carrier.

12. A method of stimulating an immune response in a pig comprising administering the composition of claim 11 to the pig in an amount effective to induce an immune response.

13. A vaccine, preferably a DIVA vaccine, comprising an effective immunizing amount of the composition of claim 11.

14. A method for preventing or ameliorating porcine epizootic diarrhea in a pig, comprising administering the PEDVdN according to claim 1 to the pig, preferably wherein the pig is a pregnant sow.
